SEQUENCE LISTING (11 sequences)

SEQ ID NO: 1 - DNA, 74 bp, unidentified
  source 1..74: mol_type = other DNA
tcagaaaatgacaaacctgtggggcgtaagtagatatgtacatatctatgatcgtgcagacgttaaaatcaggt

SEQ ID NO: 2 - DNA, 67 bp, unidentified
  source 1..67: mol_type = other DNA
ggggaagaagatgacaaccttctcgcggtacttccctgttacgtgtgtagacacaacgaatgtcatc

SEQ ID NO: 3 - DNA, 82 bp, unidentified
  source 1..82: mol_type = other DNA
ggctgacaaaatcctatctctacctcctcgtggtgtcagccggaatgtgcagtatcagtactgcatataccaacagagaggg

SEQ ID NO: 4 - DNA, 22 bp, synthetic construct
  source 1..22: mol_type = other DNA
ccggatgaagaggtatggtctt

SEQ ID NO: 5 - DNA, 21 bp, synthetic construct
  source 1..21: mol_type = other DNA
taatttctactaagtgtagat

SEQ ID NO: 6 - DNA, 43 bp, synthetic construct
  source 1..43: mol_type = other DNA
taatttctactaagtgtagatccggatgaagaggtatggtctt

SEQ ID NO: 7 - DNA, 37 bp, synthetic construct
  source 1..37: mol_type = other DNA
ctgatgagtccgtgaggacgaaacgagtaagctcgtc

SEQ ID NO: 8 - DNA, 68 bp, Hepatitis Delta Virus
  source 1..68: mol_type = genomic DNA
ggccggcatggtcccagcctcctcgctggcgccggctgggcaacatgcttcggcatggcgaatgggac

SEQ ID NO: 9 - RNA, 74 nt, unidentified
  source 1..74: mol_type = other RNA
tcagaaaatgacaaacctgtggggcgtaagtagatatgtacatatctatgatcgtgcagacgttaaaatcaggt

SEQ ID NO: 10 - RNA, 67 nt, Petromyzon marinus
  source 1..67: mol_type = genomic RNA
ggggaagaagatgacaaccttctcgcggtacttccctgttacgtgtgtagacacaacgaatgtcatc

SEQ ID NO: 11 - RNA, 82 nt, Anopheles gambiae
  source 1..82: mol_type = genomic RNA
ggctgacaaaatcctatctctacctcctcgtggtgtcagccggaatgtgcagtatcagtactgcatataccaacagagaggg

(19) United States
(12) Patent Application Publication
(10) Pub. No.: US 2026/0308579 A1
(43) Pub. Date: Oct. 8, 2026

(54) RIBOZYME SEQUENCES FOR PROCESSING RNA

(71) Applicant: INARI AGRICULTURE TECHNOLOGY, INC., Cambridge, MA (US)

(72) Inventors: Brianne Conlon, Somerville, MA (US); Scott A. Bevan, Carmel, IN (US); Tomáš Cermák, Saint Charles, IL (US)

(21) Appl. No.: 19/479,934

(22) PCT Filed: Apr. 30, 2024
(86) PCT No.: PCT/US2024/026941

§ 371 Date: Oct. 29, 2025

Related U.S. Application Data
(60) Provisional application No. 63/499,656, filed on May 2, 2023.

(51) Int. Cl. C12N 15/11; C12N 9/22; C12N 15/90

(52) U.S. Cl. CPC C12N 15/11; C12N 9/226; C12N 15/907; C12N 2310/20

(57) ABSTRACT

DNA molecules containing ribozymes which provide for product RNAs with defined 3′ termini are provided along with cells containing the DNA molecules and associated methods.

### CROSS REFERENCE TO RELATED APPLICATIONS

This International application claims benefit of provisional application U.S. Ser. No. 63/499,656, filed May 2, 2023, which is hereby incorporated herein by reference in its entirety.

### INCORPORATION OF SEQUENCE LISTING

The instant application contains a Sequence Listing which has been submitted electronically in XML file format and is hereby incorporated by reference in its entirety. The XML file, created on Apr. 24, 2024, is named “P14344WO00.xml” and is 11,130 bytes in size. The XML file, filed on May 2, 2023, in U.S. Patent Application Ser. No. 63/499,656, named “P14344US00_SequenceListing.xml” and which is 10,953 bytes in size, is also incorporated herein by reference in its entirety.

### TECHNICAL FIELD

The present disclosure relates generally to providing product RNA molecules with defined 3′ termini.

### BACKGROUND

In certain instances, product RNA molecules having a 3′ termini are desired. One potential solution for obtaining a desired product RNA with a defined 3′ terminus is to provide a precursor RNA compromising a product RNA with an autocatalytic ribozyme at its 3′ terminus. Other autocatalytic ribozymes which can provide for defined 3′ termini are provided herein.

### SUMMARY

DNA molecules comprising from 5′ to 3′ of their sense strand a promoter which is operably linked to a DNA molecule encoding a precursor RNA molecule comprising from its 5′ to 3′ end a product RNA molecule which is operably linked at its 3′ end to a 3′-terminal ribozyme comprising an RNA encoded by SEQ ID NO: 1, 2, 3, or a conservatively substituted variant thereof having autocatalytic RNA cleavage activity, wherein the product RNA molecule is heterologous to the 3′-terminal ribozyme are provided herein.

Cells which comprise the DNA molecules, including bacterial cells, plant cells, and mammalian cells, are provided herein. Also provided herein are transgenic plants comprising the DNA molecules.

Methods of delivering a product RNA to a cell comprising the step of introducing one or more of the aforementioned DNA molecules into the cell are provided.

Methods of constructing a recombinant DNA molecule encoding a precursor RNA molecule comprising insertion of a first DNA molecule comprising SEQ ID NO: 1, 2, 3, or a conservatively substituted variant thereof which encodes a ribozyme with autocatalytic RNA cleavage activity at the 3′ end of a sense strand of a second DNA molecule encoding a product RNA molecule to obtain a recombinant DNA molecule encoding the precursor RNA molecule, wherein the product RNA molecule is heterologous to the ribozyme and is operably linked to the ribozyme in the precursor RNA molecule are provided.

Methods of producing an RNA with a defined 3′ terminus in a host cell comprising introducing into the host cell a recombinant DNA comprising a promoter which is operably linked to DNA encoding a precursor RNA molecule comprising from 5′ to 3′ a product RNA molecule which is operably linked at its 3′ end to a 3′-terminal ribozyme comprising an RNA encoded by SEQ ID NO: 1, 2, 3, or a conservatively substituted variant thereof having autocatalytic RNA cleavage activity, wherein the product RNA molecule is heterologous to the 3′-terminal ribozyme and wherein the product RNA is produced in the host cell are provided.

### BRIEF DESCRIPTION OF THE DRAWINGS

FIG. 1 shows a DNA molecule comprising a promoter which drives expression of a precursor RNA molecule. The precursor RNA molecule comprises a product RNA with ribozymes at its 5′ and 3′ terminus. The product RNA is produced upon self-cleavage of the ribozymes at its 5′ and 3′ terminus.

FIG. 2 shows a DNA molecule comprising a promoter which drives expression of a precursor RNA molecule where the product RNA is a guide RNA comprising a direct repeat (DR) element and a spacer element. product RNA is produced upon self-cleavage of the ribozymes at its 5′ and 3′ terminus.

FIG. 3A, B shows the frequency of: (3A) obtaining Indels (DNA insertions or deletions) at a target site in soybean treated with various controls and vectors as indicated; and (3B) obtaining Top 2 alleles in soybean treated with various controls and vectors as indicated.

### DETAILED DESCRIPTION

Unless defined otherwise, all technical and scientific terms used above have the same meaning as commonly understood by one of ordinary skill in the art to which embodiments of the present disclosure pertain.

Unless otherwise stated, nucleic acid sequences in the text of this specification are given, when read from left to right, in the 5′ to 3′ direction. Nucleic acid sequences may be provided as DNA or as RNA, as specified; disclosure of one necessarily defines the other, as well as necessarily defines the exact complements, as is known to one of ordinary skill in the art.

The terms “a,” “an,” and “the” include both singular and plural referents.

The term “and/or” where used herein is to be taken as specific disclosure of each of the two specified features or components with or without the other. Thus, the term and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone).

As used herein, the phrases “endogenous sequence,” “endogenous gene,” “endogenous DNA,” “endogenous polynucleotide,” and the like refer to the native form of a polynucleotide, gene or polypeptide in its natural location in the organism or in the genome of an organism.

As used herein, the phrase “defined 3′ terminus” refers to a 3′ terminus of an RNA molecule which terminates at a pre-selected and desired 3′ ribonucleotide residue. In an exemplary embodiment, a defined 3′ terminus of a Cas12 guide RNA would terminate at its 3′ terminus in the last nucleotide of the spacer RNA and would not comprise additional, extraneous ribonucleotide residues at its 3′ terminus.

As used herein, the term “exemplary” refers to an example, an instance, or an illustration, and does not indicate a most preferred embodiment unless otherwise stated.

As used herein, a “heterologous” agent or molecule refers: (i) to any agent or molecule that is not found in a wild-type, untreated, or naturally occurring composition, eukaryotic cell, or plant cell; and/or (ii) to a polynucleotide or peptide sequence located in, e.g., a genome or a vector, in a context other than that in which the sequence occurs in nature. For example, a promoter that is operably linked to a gene other than the gene that the promoter is operably linked to in nature is a heterologous promoter. Similarly, a product RNA molecule is heterologous to the 3′-terminal ribozyme when the product RNA molecule is not operably linked to the 3′-terminal ribozyme in nature.

As used herein, the terms “include,” “includes,” and “including” are to be construed as at least having the features to which they refer while not excluding any additional unspecified features.

The phrase “operably linked” refers to a juxtaposition wherein the components so described are in a relationship permitting them to function in their intended manner. For instance, a promoter is operably linked to a coding sequence if the promoter provides for transcription or expression of the coding sequence. When the phrase “operably linked” is used in the context of a precursor RNA comprising a 3′-terminal ribozyme which is operably linked to the 3′ end of a product RNA, it refers to a covalent linkage of the 3′-terminal ribozyme to the product RNA in the precursor RNA that results in a product RNA with a desired 3′ terminus upon self-cleavage of the precursor RNA. Similarly, DNA encoding a 3′-terminal ribozyme is operably linked to the 3′ end of DNA encoding a product RNA when the precursor RNA encoded by the DNAs comprises a covalent linkage of the 3′-terminal ribozyme to the product RNA that results in a product RNA with a desired 3′ terminus upon self-cleavage of the precursor RNA.

As used herein, the phrase “plant cell” can refer either to a plant cell having a plant cell wall or to a plant cell protoplast lacking a plant cell wall.

A “recombinant adenoviral vector” refers to a polynucleotide vector comprising one or more heterologous sequences (i.e., nucleic acid sequence not of adenovirus origin) that are flanked by at least one adenovirus inverted terminal repeat sequence (ITRs). In some embodiments, the recombinant nucleic acid is flanked by two inverted terminal repeat sequences (ITRs). Such recombinant viral vectors can be replicated and packaged into infectious viral particles when present in a host cell that is expressing essential adenovirus genes deleted from the recombinant viral genome (e.g., E1 genes, E2 genes, E4 genes, etc.). When a recombinant viral vector is incorporated into a larger polynucleotide (e.g., in a chromosome or in another vector such as a plasmid used for cloning or transfection), men the recombinant viral vector may be referred to as a “pro-vector” which can be “rescued” by replication and encapsidation in the presence of adenovirus packaging functions. A recombinant viral vector can be in any of a number of forms, including, but not limited to, plasmids, linear artificial chromosomes, complexed with lipids, encapsulated within liposomes, and encapsidated in a viral particle, for example, an adenovirus particle. A recombinant viral vector can be packaged into an adenovirus virus capsid to generate a “recombinant adenoviral particle.”

A “recombinant lentivirus vector” refers to a polynucleotide vector comprising one or more heterologous sequences (i.e., nucleic acid sequence not of lentivirus origin) that are flanked by at least one lentivirus terminal repeat sequences (LTRs). In some embodiments, the recombinant nucleic acid is flanked by two lentiviral terminal repeat sequences (LTRs). Such recombinant viral vectors can be replicated and packaged into infectious viral particles when present in a host cell that has been infected with suitable helper functions. A recombinant lentiviral vector can be packaged into a lentivirus capsid to generate a “recombinant lentiviral particle.”

A “recombinant herpes simplex vector (recombinant HSV vector)” or “HSV vector” refers to a polynucleotide vector comprising one or more heterologous sequences (i.e., nucleic acid sequence not of HSV origin) that are flanked by HSV terminal repeat sequences. Such recombinant viral vectors can be replicated and packaged into infectious viral particles when present in a host cell that has been infected with suitable helper functions. When a recombinant viral vector is incorporated into a larger polynucleotide (e.g., in a chromosome or in another vector such as a plasmid used for cloning or transfection), then the recombinant viral vector may be referred to as a “pro-vector” which can be “rescued” by replication and encapsidation in the presence of HSV packaging functions. A recombinant viral vector can be in any of a number of forms, including, but not limited to, plasmids, linear artificial chromosomes, complexed with lipids, encapsulated within liposomes, and encapsidated in a viral particle, for example, an HSV particle. A recombinant viral vector can be packaged into an HSV capsid to generate a “recombinant herpes simplex viral particle.”

As used herein, the phrase “3′-terminal ribozyme” refers to a ribozyme comprising an RNA encoded by SEQ ID NO: 1, 2, 3, or a conservatively substituted variant thereof having autocatalytic RNA cleavage activity.

To the extent to which any of the preceding definitions is inconsistent with definitions provided in any patent or non-patent reference incorporated herein by reference, any patent or non-patent reference cited herein, or in any patent or non-patent reference found elsewhere, it is understood that the preceding definition will be used herein.

The present disclosure describes DNA molecules useful for production of product RNAs with defined 3′ termini in host cells. Such product RNAs include guide RNA (gRNA) molecules for CRISPR/CAS gene-editing systems (e.g., type V systems using a Cas12 nuclease and corresponding gRNAs recognized by the Cas12 nuclease), an siRNA, a miRNA, an aptamer, or a viral RNA replicon.

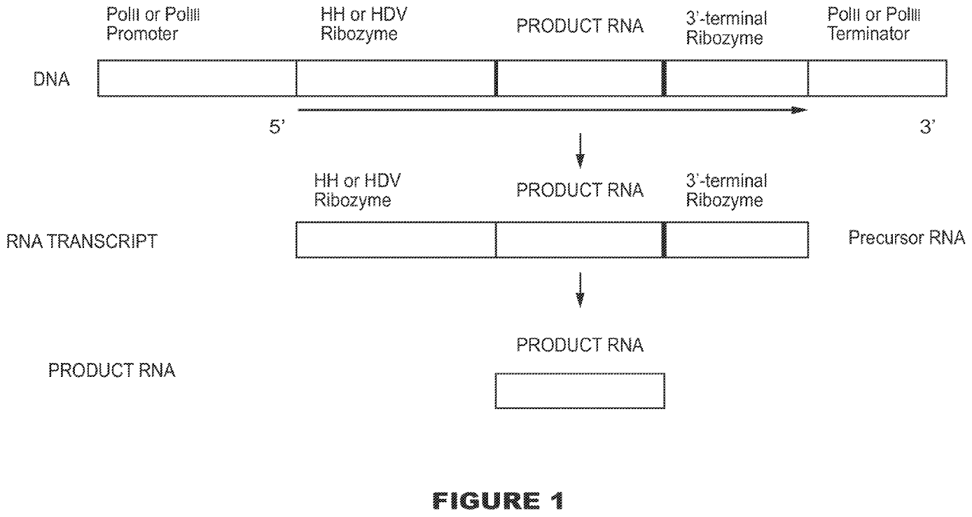

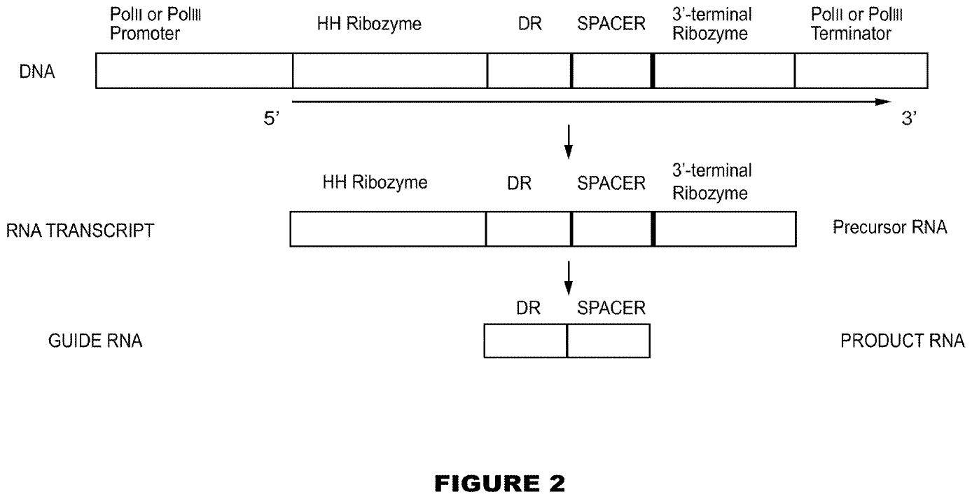

In certain embodiments, a single product RNAs can be produced from single precursor RNA under control of a single promoter, where the precursor RNA comprises a product RNA which is operably linked a 3′-terminal ribozyme. Exemplary illustrations of such precursor and product RNA molecules are set forth in FIGS. 1 and 2. In other embodiments, multiple product RNAs can also be produced from single precursor RNA under control of a single promoter. In certain embodiments, each product RNA within the single precursor RNA can comprise a 3′-terminal ribozyme which is operably linked to the 3′ terminus of each product RNA in the precursor RNA.

Disclosed herein are 3′-terminal ribozymes which are particularly useful for providing biologically and/or biochemically active product RNA molecules. In certain embodiments the 3′ terminal ribozymes comprise an RNA encoded by SEQ ID NO: 1, 2, 3, or a conservatively substituted variant thereof having autocatalytic RNA cleavage activity. In certain embodiments, a conservatively substituted variant of an RNA encoded by SEQ ID NO: 1, 2, or 3 can comprise an RNA molecule encoded by DNA molecules having at least 95%, 97%, or 99% sequence identity to SEQ ID NO: 1, 2, or 3. In certain embodiments, a conservatively substituted variant of an RNA encoded by SEQ ID NO: 1, 2, or 3 can comprise an RNA molecule having at least 95%, 97%, or 99% sequence identity to SEQ ID NO: 9, 10, or 11. In certain embodiments, a conservatively substituted variant of an RNA encoded by SEQ ID NO: 1, 2, or 3 can comprise an RNA molecule wherein one, two, three, or more purine residues in SEQ ID NO: 9, 10, or 11 are replaced with a distinct purine residue (e.g., an adenine is replaced with a guanine residue or vice versa). In certain embodiments, a conservatively substituted variant of an RNA encoded by SEQ ID NO: 1, 2, or 3 can comprise an RNA molecule wherein one, two, three, or more pyrimidine residues in SEQ ID NO: 9, 10, or 11 are replaced with a distinct pyrimidine residue (e.g., a cytosine is replaced with a uracil residue or vice versa). In certain embodiments, a conservatively substituted variant of an RNA encoded by SEQ ID NO: 1, 2, or 3 can comprise an RNA molecule wherein a purine and a pyrimidine residue in SEQ ID NO: 9, 10, or 11 which form a hydrogen-bonded base pair in the folded form of SEQ ID NO: 9, 10, or 11 which exhibits autocatalytic RNA cleavage is replaced by another purine and a pyrimidine residue which can form a hydrogen-bonded base pair.

In certain embodiments, the 3′-terminal ribozyme comprises the RNA encoded by SEQ ID NO: 1, the RNA molecule of SEQ ID NO: 9, or a conservatively substituted variant thereof. This RNA molecule is a member of a family of more than 200 “hatchet” ribozymes having a conserved RNA secondary structure comprising 4 base-paired substructures P1, P2, P3, and P4 (Li et al. 2015. RNA 21:1845-1851; doi/10.1261/rna.052522.115). In certain embodiments, conservatively substituted variants of the RNA encoded by SEQ ID NO: 1 or the RNA molecule of SEQ ID NO: 9 can comprise compensatory substitutions where the wild-type RNA residues that base-pair are substituted with non-wild type RNA residues which can base pair.

In certain embodiments, the 3′-terminal ribozyme comprises the RNA encoded by SEQ ID NO: 2 or 3, the RNA molecule of SEQ ID NO: 10 or 11, or a conservatively substituted variant thereof. These RNA molecules are members of a family of ribozymes related to the hepatitis delta virus (HDV) ribozyme which comprise six invariant nucleotides and have a conserved RNA secondary structure comprising 4 base-paired substructures P1, P2, P3, and P4 (Webb et al. Science. 2009. 326 (5955): 953; doi: 10.1126/science.1178084). In certain embodiments, conservatively substituted variants of the RNA encoded by SEQ ID NO: 2 or 3 or the RNA molecule of SEQ ID NO: 10 or 11 can comprise compensatory substitutions where the wild-type RNA residues that base-pair are substituted with non-wild type RNA residues which can base pair.

In certain embodiments, the 5′ terminus of any of the aforementioned 3′-terminal ribozymes is operably linked to the 3′ terminus of a product RNA comprising a guide RNA for a type V Cas RNA dependent endonuclease (RDE). Type V guide RNAs comprising from their 5′ to 3′ end a direct repeat (DR) element which can be bound by the type V Cas RDE and a spacer element which is operably linked to the 5′ end of the 3-terminal ribozyme (e.g. as illustrated in FIG. 2). The exemplary Type V Cas RDE Cpf1 (i.e., Cas12a) and corresponding guide RNAs and PAM sites are disclosed in US Patent Application Publication 2016/0208243 A1, which is incorporated herein by reference in its entirety. In the Type V gRNA, the DR element comprises the RNA which is recognized by the Type V RDE and the spacer element comprises the RNA sequence which hybridizes to the target cleavage site in the target DNA that is adjacent to a protospacer adjacent motif (PAM). T-rich PAM sites (e.g., 5′-TTN or 5′-TTTV, where “V” is A, C, or G) are typically targeted for design of crRNAs or sgRNAs used with type V RDE. An exemplary DR element for a type V Cas RDE is the DR encoded by SEQ ID NO: 5.

In certain embodiments, the 5′ terminus of any of the aforementioned 3′-terminal ribozymes is operably linked to the 3′ terminus of a product RNA comprising a guide RNA for a type II Cas RNA dependent endonuclease (RDE). In certain embodiments, precursor RNAs can comprise type II guide RNAs comprising from their 5′ end a spacer element and a direct repeat (DR) element which can be bound by the type II Cas RDE and which is operably linked to the 5′ end of the 3-terminal ribozyme. The type II guide RNAs comprising from their 5′ end a spacer element and a direct repeat (DR) element are typically duplexed with corresponding tracrRNAs to effect cleavage of a target nucleic acid. In other embodiments, the precursor RNAs can comprise type II single guide RNAs comprising from their 5′ end a spacer element, a direct repeat (DR) element which can be bound by the type II Cas RDE, and a covalently-linked tracrRNA which is operably linked to the 5′ end of the 3-terminal ribozyme. The exemplary Type II Cas RDE (i.e., Cas9) and corresponding duplexed and single guide RNAs and PAM sites are disclosed in US Patent Application Publication 2015/0082478 A1, the entire specification of which is incorporated herein by reference in its entirety. In the Type II gRNA, the DR element comprises the RNA which is recognized by the Type II RDE and the spacer element comprises the RNA sequence which hybridizes to the target cleavage site in the target DNA that is adjacent to a protospacer adjacent motif (PAM). Examples of PAM sequences for type II Cas nuclease systems include 5′-NGG (Streptococcus pyogenes), 5′-NNAGAA (Streptococcus thermophilus CRISPR1), 5′-NGGNG (Streptococcus thermophilus CRISPR3), 5′-NNGRRT or 5′-NNGRR (Staphylococcus aureus Cas9, SaCas9), and 5′-NNNGATT (Neisseria meningitidis).

Spacer elements that can be used with the aforementioned type V and type II Cas RDE will typically comprise at least 16, 17, or 18 nucleotides that are complementary to the target nucleic acid. In certain embodiments, at least 16 or 17 nucleotides of spacer sequence are required by a type II RDE (e.g., Cas9) for DNA cleavage to occur. In certain embodiments, at least 16 nucleotides of spacer sequence are needed to achieve detectable DNA cleavage and at least 18 nucleotides of spacer sequence were reported necessary for efficient DNA cleavage in vitro for CpfI RDE (i.e., a type V RDE; see Zetsche et al. (2015) Cell, 163:759-77. In practice, spacer RNA sequences are generally designed to have a length of 17-24 nucleotides (frequently 19, 20, or 21 nucleotides) and exact complementarity (i.e., perfect base-pairing) to the targeted gene or nucleic acid sequence; guide RNAs having less than 100% complementarity to the target sequence can be used (e.g., a gRNA with a length of 20 nucleotides and 1-4 mismatches to the target sequence) but can increase the potential for off-target effects.

Exemplary illustrations of DNA molecules which can be used to produce precursor and product RNAs are set forth in FIGS. 1 and 2. In certain embodiments, expression of the precursor RNA is provided by an RNA polymerase II (RNA polII) promoter paired with an RNA polymerase II terminator (e.g., “polyadenylation signal”) which are selected for activity in a desired host cell (e.g., a plant or mammalian cell). In certain embodiments, the promoter operably linked to one or more polynucleotides is a constitutive promoter that drives gene expression in eukaryotic cells (e.g., plant cells). In certain embodiments, the promoter drives gene expression in the nucleus or in an organelle such as a chloroplast or mitochondrion. Examples of constitutive promoters for use in plants include a CaMV 35S promoter as disclosed in U.S. Pat. Nos. 5,858,742 and 5,322,938, a rice actin promoter as disclosed in U.S. Pat. No. 5,641,876, a maize chloroplast aldolase promoter as disclosed in U.S. Pat. No. 7,151,204, and the nopaline synthase (NOS) and octopine synthase (OCS) promoters from Agrobacterium tumefaciens. In certain embodiments, the promoter operably linked to one or more polynucleotides encoding elements of a genome-editing system is a promoter from figwort mosaic virus (FMV), a RUBISCO promoter, or a pyruvate phosphate dikinase (PPDK) promoter, which is active in photosynthetic tissues. Nonlimiting examples of eukaryotic RNA polII promoters (promoters functional in a eukaryotic cell including a mammalian cell) include EF1a, those from cytomegalovirus (CMV) immediate early, herpes simplex virus (HSV) thymidine kinase, early and late SV40, long terminal repeats (LTRs) from retrovirus, and mouse metallothionein-I promoters. Useful 3′ elements that can be used for plant expression with an RNA pollI promoter include: Agrobacterium tumefaciens nos 3′, tml 3′, tmr 3′, tms 3′, ocs 3′, and tr7 3′ elements disclosed in U.S. Pat. No. 6,090,627, incorporated herein by reference, and 3′ elements from plant genes such as the heat shock protein 17, ubiquitin, and fructose-1,6-biphosphatase genes from wheat (Triticum aestivum), and the glutelin, lactate dehydrogenase, and beta-tubulin genes from rice (Oryza sativa), disclosed in US Patent Application Publication 2002/0192813 A1. Useful 3′ elements that can be used for mammalian cell expression with an RNA polII promoter include: human growth hormone (hGH) terminator, rabbit beta-globin (rb globin) terminator, and an SV40 polyA signal. All of the patent publications referenced in this paragraph are incorporated herein by reference in their entirety.

In certain embodiments, the promoter is an RNA polymerase III (RNA polIII) promoter operably linked to a nucleotide sequence encoding one or more precursor RNAs operably linked to the 3′-terminal ribozyme. In certain embodiments, the RNA polymerase III promoter is a plant U6 spliceosomal RNA promoter, which can be native to the genome of the plant cell or from a different species, e.g., a U6 promoter from maize, tomato, or soybean such as those disclosed U.S. Patent Application Publication 2017/0166912, or a homologue thereof. In acarian embodiments, such a promoter is operably linked to DNA sequence encoding a first RNA molecule including a Type V gRNA followed by an operably linked 3′ terminal ribozyme and a suitable 3′ element such as a U6 poly-T terminator. In another embodiment, the RNA polymerase III promoter is a plant U3, 7SL (signal recognition particle RNA), U2, or U5 promoter, or chimerics thereof, e.g., as described in U.S. Patent Application Publication 20170166912. In certain embodiments useful for expression in mammalian cells, the polIII promoter is a H. sapiens U6 (HsU6) promoter. In certain embodiments, the promoter operably linked to one or more polynucleotides is a constitutive promoter that drives gene expression in eukaryotic cells (e.g., plant cells). In certain embodiments, the promoter drives gene expression in the nucleus or in an organelle such as a chloroplast or mitochondrion. All of the patent publications referenced in this paragraph are incorporated herein by reference in their entirety.

Non-limiting examples of cells (target cells) include: a prokaryotic cell, eukaryotic cell, a bacterial cell, an archaeal cell, a cell of a single-cell eukaryotic organism, a protozoa cell, a cell from a plant (e.g., cells from plant crops, fruits, vegetables, grains, soy bean, corn, maize, wheat, seeds, tomatos, rice, cassava, sugarcane, pumpkin, hay, potatos, cotton, Brassica sp. including oilseed rape, sorghum, sugarbeet, cannabis, tobacco, flowering plants, conifers, gymnosperms, angiosperms, ferns, clubmosses, hornworts, liverworts, mosses, dicotyledons, monocotyledons, etc.), an algal cell, (e.g., Botryococcus braunii, Chlamydomonas reinhardtii, Nannochloropsis gaditana, Chlorella pyrenoidosa, Sargassum patens, C. agardh, and the like), seaweeds (e.g., kelp) a fungal cell (e.g., a yeast cell, a cell from a mushroom), an animal cell, a cell from an invertebrate animal (e.g., fruit fly, cnidarian, echinoderm, nematode, etc.), a cell from a vertebrate animal (e.g., fish, amphibian, reptile, bird, mammal), a cell from a mammal (e.g., an ungulate (e.g., a pig, a cow, a goat, a sheep); a rodent (e.g., a rat, a mouse); a non-human primate; a human; a feline (e.g., a cat); a canine (e.g., a dog); etc.), and the like. In some embodiments, the cell is a cell that does not originate from a natural organism (e.g., the cell can be a synthetically made cell; also referred to as an artificial cell).

A cell can be an in vitro cell (e.g., established cultured cell line). A cell can be an ex vivo cell (cultured cell from an individual). A cell can be an in vivo cell (e.g., a cell in an individual). A cell can be an isolated cell. A cell can be a cell inside of an organism. A cell can be an organism. A cell can be a cell in a cell culture (e.g., in vitro cell culture). A cell can be one of a collection of cells. A cell can be a prokaryotic cell or derived from a prokaryotic cell. A cell can be a bacterial cell or can be derived from a bacterial cell. A cell can be an archaeal cell or derived from an archaeal cell. A cell can be a eukaryotic cell or derived from a eukaryotic cell. A cell can be a plant cell or derived from a plant cell. A cell can be an animal cell or derived from an animal cell. A cell can be an invertebrate cell or derived from an invertebrate cell. A cell can be a vertebrate cell or derived from a vertebrate cell. A cell can be a mammalian cell or derived from a mammalian cell. A cell can be a rodent cell or derived from a rodent cell. A cell can be a human cell or derived from a human cell. A cell can be a microbial cell or derived from a microbe cell. A cell can be a fungi cell or derived from a fungi cell. A cell can be an insect cell. A cell can be an arthropod cell. A cell can be a protozoan cell. A cell can be a helminth cell.

Suitable cells include a stem cell (e.g., an embryonic stem (ES) cell, an induced pluripotent stem (iPS) cell; a germ cell (e.g., an oocyte, a sperm, an oogonia, a spermatogonia, etc.); a somatic cell, e.g., a fibroblast, an oligodendrocyte, a glial cell, a hematopoietic cell, a neuron, a muscle cell, a bone cell, a hepatocyte, a pancreatic cell, etc.

Suitable cells include human embryonic stem cells, fetal cardiomyocytes, myofibroblasts, mesenchymal stem cells, autotransplanted expanded cardiomyocytes, adipocytes, totipotent cells, pluripotent cells, blood stem cells, myoblasts, adult stem cells, bone marrow cells, mesenchymal cells, embryonic stem cells, parenchymal cells, epithelial cells, endothelial cells, mesothelial cells, fibroblasts, osteoblasts, chondrocytes, exogenous cells, endogenous cells, stem cells, hematopoietic stem cells, bone-marrow derived progenitor cells, myocardial cells, skeletal cells, fetal cells, undifferentiated cells, multi-potent progenitor cells, unipotent progenitor cells, monocytes, cardiac myoblasts, skeletal myoblasts, macrophages, capillary endothelial cells, xenogenic cells, allogenic cells, and post-natal stem cells.

In some embodiments, the cell is an immune cell, a neuron, an epithelial cell, and endothelial cell, or a stem cell. In some embodiments, the immune cell is a T cell, a B cell, a monocyte, a natural killer cell, a dendritic cell, or a macrophage. In some embodiments, the immune cell is a cytotoxic T cell. In some embodiments, the immune cell is a helper T cell. In some embodiments, the immune cell is a regulatory T cell (Treg).

In some embodiments, the cell is a stem cell. Stem cells include adult stem cells. Adult stem cells are also referred to as somatic stem cells. Adult stem cells are resident in differentiated tissue, but retain the properties of self-renewal and ability to give rise to multiple cell types, usually cell types typical of the tissue in which the stem cells are found. Numerous examples of somatic stem cells are known to those of skill in the art, including muscle stem cells; hematopoietic stem cells; epithelial stem cells; neural stem cells; mesenchymal stem cells; mammary stem cells; intestinal stem cells; mesodermal stem cells; endothelial stem cells; olfactory stem cells; neural crest stem cells; and the like.

Stem cells of interest include mammalian stem cells, where the term “mammalian” refers to any animal classified as a mammal, including humans; non-human primates; domestic and farm animals; and zoo, laboratory, sports, or pet animals, such as dogs, horses, cats, cows, mice, rats, rabbits, etc. In some embodiments, the stem cell is a human stem cell. In some embodiments, the stem cell is a rodent (e.g., a mouse; a rat) stem cell. In some embodiments, the stem cell is a non-human primate stem cell. Stem cells can express one or more stem cell markers, e.g., SOX9, KRT19, KRT7, LGR5, CA9, FXYD2, CDH6, CLDN18, TSPAN8, BPIFB1, OLFM4, CDH17, and PPARGC1A.

In some embodiments, the stem cell is a hematopoietic stem cell (HSC). HSCs are mesoderm-derived cells that can be isolated from bone marrow, blood, cord blood, fetal liver and yolk sac. HSCs are characterized as CD34+ and CD3+. HSCs can repopulate the erythroid, neutrophil, macrophage, megakaryocyte and lymphoid hematopoietic cell lineages in vivo. In vitro, HSCs can be induced to undergo at least some self-renewing cell divisions and can be induced to differentiate to the same lineages as is seen in vivo. As such, HSCs can be induced to differentiate into one or more of erythroid cells, megakaryocytes, neutrophils, macrophages, and lymphoid cells.

In other embodiments, the stem cell is a neural stem cell (NSC). NSCs are capable of differentiating into neurons, and glia (including oligodendrocytes, and astrocytes). A neural stem cell is a multipotent stem cell which is capable of multiple divisions, and under specific conditions can produce daughter cells which are neural stem cells, or neural progenitor cells that can be neuroblasts or glioblasts, e.g., cells committed to become one or more types of neurons and glial cells respectively. Methods of obtaining NSCs are known in the art.

In other embodiments, the stem cell is a mesenchymal stem cell (MSC). MSCs originally derived from the embryonal mesoderm and isolated from adult bone marrow, can differentiate to form muscle, bone, cartilage, fat, marrow stroma, and tendon. Methods of isolating MSC are known in the art; and any known method can be used to obtain MSC. See, e.g., U.S. Pat. No. 5,736,396, which describes isolation of human MSC.

In certain optional embodiments, the methods disclosed herein are not processes for modifying the germ line genetic identity of human beings. In certain optional embodiments, the methods disclosed herein are not processes for modifying the genetic identity of animals which are likely to cause them suffering without any substantial medical benefit to man or animal, and also animals resulting from such processes. In certain optional embodiments, the methods disclosed herein are not methods for treatment of the human or animal body by surgery or therapy. In a certain optional embodiments, the cells disclosed herein are not human embryos. In certain optional embodiments, the cells disclosed herein are not the human body, at the various stages of its formation and development.

A cell is in some embodiments a plant cell. A plant cell can be a cell of a monocotyledon. A cell can be a cell of a dicotyledon. For example, the cell can be a cell of a major agricultural plant, e.g., Barley, Beans (Dry Edible), Canola, Corn, Cotton (Pima), Cotton (Upland), Flaxseed, Hay (Alfalfa), Hay (Non-Alfalfa), Oats, Peanuts, Rice, Sorghum, Soybeans, Sugarbeets, Sugarcane, Sunflowers (Oil), Sunflowers (Non-Oil), Sweet Potatoes, Tobacco (Burley), Tobacco (Flue-cured), Tomatoes, Wheat (Durum), Wheat (Spring), Wheat (Winter), and the like. As another example, the cell is a cell of a vegetable crops which include but are not limited to, e.g., alfalfa sprouts, aloe leaves, arrow root, arrowhead, artichokes, asparagus, bamboo shoots, banana flowers, bean sprouts, beans, beet tops, beets, bittermelon, bok choy, broccoli, broccoli rabe (rappini), brussels sprouts, cabbage, cabbage sprouts, cactus leaf (nopales), calabaza, cardoon, carrots, cauliflower, celery, chayote, Chinese artichoke (crosnes), Chinese cabbage, Chinese celery, Chinese chives, choy sum, chrysanthemum leaves (tung ho), collard greens, corn stalks, corn-sweet, cucumbers, daikon, dandelion greens, dasheen, dau mue (pea tips), donqua (winter melon), eggplant, endive, escarole, fiddle head ferns, field cress, frisee, gai choy (chinese mustard), gailon, galanga (siam, thai ginger), garlic, ginger root, gobo, greens, hanover salad greens, huauzontle, jerusalem artichokes, jicama, kale greens, kohlrabi, lamb's quarters (quilete), lettuce (bibb), lettuce (boston), lettuce (boston red), lettuce (green leaf), lettuce (iceberg), lettuce (lolla rossa), lettuce (oak leaf-green), lettuce (oak leaf-red), lettuce (processed), lettuce (red leaf), lettuce (romaine), lettuce (ruby romaine), lettuce (russian red mustard), linkok, lo bok, long beans, lotus root, mache, maguey (agave) leaves, malanga, mesculin mix, mizuna, moap (smooth luffa), moo, moqua (fuzzy squash), mushrooms, mustard, nagaimo, okra, ong choy, onions green, opo (long squash), ornamental corn, ornamental gourds, parsley, parsnips, peas, peppers (bell type), peppers, pumpkins, radicchio, radish sprouts, radishes, rape greens, rape greens, rhubarb, romaine (baby red), rutabagas, salicornia (sea bean), sinqua (angled/ridged luffa), spinach, squash, straw bales, sugarcane, sweet potatoes, swiss chard, tamarindo, taro, taro leaf, taro shoots, tatsoi, tepeguaje (guaje), tindora, tomatillos, tomatoes, tomatoes (cherry), tomatoes (grape type), tomatoes (plum type), tumeric, turnip tops greens, turnips, water chestnuts, yampi, yams (names), yu choy, yuca (cassava), and the like.

A cell is in some embodiments an arthropod cell. For example, the cell can be a cell of a suborder, a family, a sub-family, a group, a sub-group, or a species of, e.g., Chelicerata, Myriapodia, Hexipodia, Arachnida, Insecta, Archaeognatha, Thysanura, Palaeoptera, Ephemeroptera, Odonata, Anisoptera, Zygoptera, Neoptera, Exopterygota, Plecoptera, Embioptera, Orthoptera, Zoraptera, Dermaptera, Dictyoptera, Notoptera, Grylloblattidae, Mantophasmatidae, Phasmatodea, Blattaria, Isoptera, Mantodea, Parapneuroptera, Psocoptera, Thysanoptera, Phthiraptera, Hemiptera, Endopterygota or Holometabola, Hymenoptera, Coleoptera, Strepsiptera, Raphidioptera, Megaloptera, Neuroptera, Mecoptera, Siphonaptera, Diptera, Trichoptera, or Lepidoptera.

A cell is in some embodiments an insect cell. For example, in some embodiments, the cell is a cell of a mosquito, a grasshopper, a true bug, a fly, a flea, a bee, a wasp, an ant, a louse, a moth, or a beetle.

Methods of introducing a nucleic acid (e.g., one or more nucleic acids encoding a product RNA molecule which is operably linked at its 3′ end to a 3′-terminal ribozyme, and the like) into a host cell are known in the art, and any convenient method can be used to introduce a nucleic acid (e.g., an expression construct) into a cell. Suitable methods include e.g., viral infection, transfection, lipofection, electroporation, calcium phosphate precipitation, polyethyleneimine (PEI)-mediated transfection, DEAE-dextran mediated transfection, liposome-mediated transfection, particle gun technology, calcium phosphate precipitation, direct microinjection, nanoparticle-mediated nucleic acid delivery, and the like. Nucleic acids may be provided to the cells using well-developed transfection techniques; see, e.g., Angel and Yanik (2010) PLOS ONE 5 (7): el 1756, and the commercially available TransMessenger® reagents from Qiagen, STEMFECT™ RNA Transfection Kit from Stemgent, and TRANSIT®-mRNA Transfection Kit from Mirus Bio LLC. See also Beumer et al. (2008) PNAS 105 (50): 19821-19826.

Retroviruses, for example, lentiviruses and recombinant lentivirus vectors, are suitable for use in methods of the present disclosure. Commonly used retroviral vectors are “defective”, e.g., unable to produce viral proteins required for productive infection. Rather, replication of the vector requires growth in a packaging cell line. To generate viral particles comprising nucleic acids of interest, the retroviral nucleic acids comprising the nucleic acid are packaged into viral capsids by a packaging cell line. Different packaging cell lines provide a different envelope protein (ecotropic, amphotropic or xenotropic) to be incorporated into the capsid, this envelope protein determining the specificity of the viral particle for the cells (ecotropic for murine and rat; amphotropic for most mammalian cell types including human, dog and mouse; and xenotropic for most mammalian cell types except murine cells). The appropriate packaging cell line may be used to ensure that the cells are targeted by the packaged viral particles. Methods of introducing subject vector expression vectors into packaging cell lines and of collecting the viral particles that are generated by the packaging lines are well known in the art. Nucleic acids can also introduced by direct micro-injection (e.g., injection of RNA).

Introducing the recombinant expression vector into cells can occur in any culture media and under any culture conditions that promote the survival of the cells. Introducing the recombinant expression vector into a target cell can be carried out in vivo or ex vivo. Introducing the recombinant expression vector into a target cell can be carried out in vitro.

Methods of introducing exogenous nucleic acids into plant cells are well known in the art. Such plant cells are considered “transformed,” as defined above. Suitable methods include viral infection (such as double stranded DNA viruses including geminiviruses), transfection, conjugation, protoplast fusion, electroporation, particle gun technology, calcium phosphate precipitation, direct microinjection, silicon carbide whiskers technology, Agrobacterium-mediated transformation and the like. The choice of method is generally dependent on the type of cell being transformed and the circumstances under which the transformation is taking place (e.g., in vitro, ex vivo, or in vivo).

Bacterially-mediated transformation methods based upon the soil bacterium Agrobacterium tumefaciens or other bacteria (e.g., Rhizobium sp., Sinorhizobium sp., Mesorhizobium sp., Bradyrhizobium sp., Azobacter sp., Phyllobacterium sp. see, e.g., Broothaerts et al. (2005) Nature, 433:629-633) are particularly useful for introducing an exogenous nucleic acid molecule into a vascular plant. The wild type form of Agrobacterium contains a Ti (tumor-inducing) plasmid that directs production of tumorigenic crown gall growth on host plants. Transfer of the tumor-inducing T-DNA region of the Ti plasmid to a plant genome requires the Ti plasmid-encoded virulence genes as well as T-DNA borders, which are a set of direct DNA repeats that delineate the region to be transferred. An Agrobacterium-based vector is a modified form of a Ti plasmid, in which the tumor inducing functions are replaced by the nucleic acid sequence of interest to be introduced into the plant host.

Agrobacterium-mediated transformation generally employs cointegrate vectors or binary vector systems, in which the components of the Ti plasmid are divided between a helper vector, which resides permanently in the Agrobacterium host and carries the virulence genes, and a shuttle vector, which contains the gene of interest bounded by T-DNA sequences. A variety of binary vectors are well known in the art and are commercially available, for example, from Clontech (Palo Alto, Calif.). Methods of coculturing Agrobacterium with cultured plant cells or wounded tissue such as leaf tissue, root explants, hypocotyledons, stem pieces or tubers, for example, also are well known in the art. See, e.g., Glick and Thompson, (eds.), Methods in Plant Molecular Biology and Biotechnology, Boca Raton, Fla.: CRC Press (1993).

Microprojectile-mediated transformation also can be used to produce a subject transgenic plant. This method, first described by Klein et al. (Nature 327:70-73 (1987)), relies on microprojectiles such as gold or tungsten that are coated with the desired nucleic acid molecule by precipitation with calcium chloride, spermidine or polyethylene glycol. The microprojectile particles are accelerated at high speed into an angiosperm tissue using a device such as the BIOLISTIC PD-1000 (Biorad; Hercules Calif.). A nucleic acid of the present disclosure (e.g., a nucleic acid (e.g., a recombinant expression vector) comprising a nucleotide sequence encoding precursor RNA comprising a 3′-terminal ribozyme of the present disclosure) may be introduced into a plant in a manner such that the nucleic acid is able to enter a plant cell(s), e.g., via an in vivo or ex vivo protocol. By “in vivo,” it is meant in the nucleic acid is administered to a living body of a plant e.g., infiltration. By “ex vivo” it is meant that cells or explants are modified outside of the plant, and then such cells or organs are regenerated to a plant. A number of vectors suitable for stable transformation of plant cells or for the establishment of transgenic plants have been described, including those described in Weissbach and Weissbach, (1989) Methods for Plant Molecular Biology Academic Press, and Gelvin et al., (1990) Plant Molecular Biology Manual, Kluwer Academic Publishers. Specific examples include those derived from a Ti plasmid of Agrobacterium tumefaciens, as well as those disclosed by Herrera-Estrella et al. (1983) Nature 303:209, Bevan (1984) Nucl Acid Res. 12:8711-8721, Klee (1985) Bio/Technolo 3:637-642.

Alternatively, non-Ti vectors can be used to transfer the DNA into plants and cells by using free DNA delivery techniques. By using these methods transgenic plants such as wheat, rice (Christou (1991) Bio/Technology 9:957-9 and 4462) and corn (Gordon-Kamm (1990) Plant Cell 2:603-618) can be produced. An immature embryo can also be a good target tissue for monocots for direct DNA delivery techniques by using the particle gun (Weeks et al. (1993) Plant Physiol 102:1077-1084; Vasil (1993) Bio/Technolo 10:667-674; Wan and Lemeaux (1994) Plant Physiol 104:37-48 and for Agrobacterium-mediated DNA transfer (Ishida et al. (1996) Nature Biotech 14:745-750). Methods for introduction of DNA into chloroplasts are biolistic bombardment, polyethylene glycol transformation of protoplasts, and microinjection (Danieli et al Nat. Biotechnol 16:345-348, 1998; Staub et al Nat. Biotechnol 18:333-338, 2000; O'Neill et al Plant J. 3:729-738, 1993; Knoblauch et al Nat. Biotechnol 17:906-909; U.S. Pat. Nos. 5,451,513, 5,545,817, 5,545,818, and 5,576,198; in Intl. Application No. WO 95/16783; and in Boynton et al., Methods in Enzymology 217:510-536 (1993), Svab et al., Proc. Natl. Acad. Sci. USA 90:913-917 (1993), and McBride et al., Proc. Natl. Acad. Sci. USA 91:7301-7305 (1994)). Any vector suitable for the methods of biolistic bombardment, polyethylene glycol transformation of protoplasts and microinjection will be suitable as a targeting vector for chloroplast transformation. Any double stranded DNA vector may be used as a transformation vector, especially when the method of introduction does not utilize Agrobacterium.

### Embodiments

Various embodiments of the DNA molecules, cells comprising same, and associated methods are set forth in the following sets of numbered embodiments.
1. A DNA molecule comprising from 5′ to 3′ of its sense strand a promoter which is operably linked to a DNA molecule encoding a precursor RNA molecule comprising from its 5′ to 3′ end a product RNA molecule which is operably linked at its 3′ end to a 3′-terminal ribozyme comprising an RNA encoded by SEQ ID NO: 1, 2, 3, or a conservatively substituted variant thereof having autocatalytic RNA cleavage activity, wherein the product RNA molecule is heterologous to the 3′-terminal ribozyme.
2. The DNA molecule of embodiment 1, wherein the product RNA molecule comprises a guide RNA for a Cas RNA dependent endonuclease (RDE).
3. The DNA molecule of embodiment 1, wherein the product RNA molecule comprises a guide RNA for a type V Cas RNA dependent endonuclease (RDE), the guide RNA comprising from its 5′ to 3′ end a direct repeat (DR) element which can be bound by the type V Cas RDE and a spacer element which is operably linked to the DR.
4. The DNA molecule of embodiment 3, wherein the precursor RNA molecule comprising the guide RNA and the 3′-terminal ribozyme further comprises at its 5′ end a hammerhead ribozyme which is operably linked to the guide RNA.
5. The DNA molecule of embodiment 1, wherein the precursor RNA molecule comprises from its 5′ to 3′ end a hammerhead ribozyme, the guide RNA comprising a direct repeat (DR) element which can be bound by a type V Cas RDE a spacer element, and the 3′-terminal ribozyme, wherein the hammerhead ribozyme and the 3′-terminal ribozyme are operably linked to the guide RNA.
6. The DNA molecule of embodiment 1, wherein the product RNA molecule comprises a guide RNA for a type II Cas RNA dependent endonuclease (RDE), the guide RNA comprising from its 5′ to 3′ end a spacer element and direct repeat (DR) element which can be bound by the type II Cas RDE and which is operably linked to the spacer element.
7. The DNA molecule of embodiment 6, wherein the precursor RNA molecule comprising the guide RNA and the 3′-terminal ribozyme further comprises at its 5′ end a hammerhead ribozyme or a hepatitis delta virus (HDV) ribozyme which is operably linked to the guide RNA.
8. The DNA molecule of embodiment 6, wherein the guide RNA is a single gRNA wherein the product RNA further comprises a tracrRNA which is operably linked to the 3′ terminus of the direct repeat.
9. The DNA molecule of embodiment 1, wherein the product RNA molecule comprises an siRNA, an miRNA, an aptamer, or a viral RNA replicon.
10. The DNA molecule of any one of embodiment 9, wherein the precursor RNA molecule further comprises at its 5′ end hammerhead ribozyme or a hepatitis delta virus (HDV) ribozyme which is operably linked to the product RNA molecule.
11. The DNA molecule of embodiment any one of embodiments 1 to 10, wherein the DNA molecule further comprises at the 3′end of the sense strand a terminator element which is operably linked to the DNA encoding the precursor RNA molecule.
12. The DNA molecule of any one of embodiments 1 to 11, wherein the promoter and/or the terminator are recognized by a eukaryotic RNA Polymerase II, optionally wherein the eukaryotic RNA Polymerase II is a monocot or dicot plant RNA Polymerase II or optionally wherein the eukaryotic RNA Polymerase II is a mammalian RNA Polymerase II.
13. The DNA molecule of any one of embodiments 1 to 11, wherein the promoter and/or the terminator are recognized by a eukaryotic RNA Polymerase III, optionally wherein the eukaryotic RNA Polymerase III is a monocot or dicot plant RNA Polymerase III or optionally wherein the eukaryotic RNA Polymerase III is a mammalian RNA Polymerase III.
14. A cell comprising the DNA molecule of any one of embodiments 1 to 13.
15. The cell of embodiment 14, wherein the cell is a bacterial cell, a plant cell, or a mammalian cell.
16. The cell of embodiment 15, wherein the bacterial cell is an Agrobacterium, Rhizobium sp., Sinorhizobium sp., Mesorhizobium sp., Bradyrhizobium sp., Azobacter sp., or Phyllobacterium sp. and optionally wherein the DNA molecule further comprises the promoter and the terminator recognized by the eukaryotic RNA Polymerase II or RNA Polymerase III and optionally wherein the eukaryotic RNA Polymerase II or RNA Polymerase III is the monocot or dicot plant RNA Polymerase II or RNA Polymerase III.
17. The cell of embodiment 15, wherein the cell is a monocot or a dicot plant cell, optionally wherein the DNA molecule further comprises the promoter and the terminator recognized by a eukaryotic RNA Polymerase II or RNA Polymerase III and optionally wherein: (i) the eukaryotic RNA Polymerase II or RNA Polymerase III is a monocot plant RNA Polymerase II or RNA Polymerase III and the cell is a monocot plant cell; or (ii) optionally wherein the eukaryotic RNA Polymerase II or RNA Polymerase III is a dicot plant RNA Polymerase II molecule or RNA Polymerase II and the cell is a dicot plant cell.
18. The cell of embodiment 15, wherein the cell is a mammalian cell, optionally wherein the DNA molecule further comprises the promoter and the terminator recognized by a mammalian RNA Polymerase II or RNA Polymerase III; optionally wherein the mammalian cell is not directly obtained from a human embryo; and/or optionally wherein the DNA molecule is provided in a recombinant adenoviral, lentiviral, or HSV vector.
19. A method of delivering a product RNA to a cell comprising the step of introducing the DNA molecule of any one of embodiments 1 to 13 into the cell.
20. The method of embodiment 19, wherein the cell is a monocot or a dicot plant cell, optionally wherein the DNA molecule further comprises the promoter and the terminator recognized by a eukaryotic RNA Polymerase II or RNA Polymerase III and optionally wherein: (i) the eukaryotic RNA Polymerase II or RNA Polymerase III is a monocot plant RNA Polymerase II or RNA Polymerase III and the cell is a monocot plant cell; or (ii) optionally wherein the eukaryotic RNA Polymerase II or RNA Polymerase III is a dicot plant RNA Polymerase II or RNA Polymerase III and the cell is a dicot plant cell.
21. The method of embodiment 19, wherein the cell is a mammalian cell, optionally wherein the DNA molecule further comprises the promoter and the terminator recognized by the mammalian RNA Polymerase II or RNA Polymerase III and optionally wherein the mammalian cell is not directly obtained from a human embryo.
22. The method of embodiment 19, wherein the product RNA comprises an siRNA, an miRNA, an aptamer, or a viral RNA replicon.
23. The method of embodiment 19, wherein the product RNA molecule comprises a guide RNA for a Cas RNA dependent endonuclease (RDE).
24. The method of embodiment 19, wherein the product RNA comprises a guide RNA for a type V Cas RNA dependent endonuclease (RDE), the guide RNA comprising from its 5′ to 3′ end a direct repeat (DR) element which can be bound by the type V Cas RDE and a spacer element which is operably linked to the DR, optionally wherein the precursor RNA molecule comprising the guide RNA and the 3′-terminal ribozyme further comprises at its 5′ end a hammerhead ribozyme.
25. The method of embodiment 24, wherein the method further comprises introducing a polynucleotide encoding the type V Cas RNA dependent endonuclease (RDE) into the cell.
26. The method of embodiment 24 or 25, wherein the method further comprises introducing a polynucleotide encoding the type V Cas RNA dependent endonuclease (RDE) and a polynucleotide comprising a DNA donor template into the cell.
27. The method of any one of embodiments 24, 25, or 26, where the precursor RNA comprises the 5′ hammerhead ribozyme and wherein the frequency of gene editing events is increased in comparison to a control comprising a DNA molecule comprising the promoter, the 5′ hammerhead ribozyme, the guide RNA, and a hepatitis delta virus (HDV) ribozyme which is operably linked to the 3′ terminus of the guide DNA in the control RNA molecule.
28. The method of embodiment 19, wherein the product RNA comprises a guide RNA for a type II Cas RNA dependent endonuclease (RDE), the guide RNA comprising from its 5′ to 3′ end a spacer element and direct repeat (DR) element which can be bound by the type II Cas RDE and which is operably linked to the spacer element, optionally wherein the precursor RNA molecule comprising the guide RNA and the 3′-terminal ribozyme further comprises at its 5′ end a hammerhead ribozyme or a hepatitis delta virus (HDV) ribozyme.
29. The method of embodiment 28, wherein the guide RNA is a single gRNA and wherein the product RNA further comprises a tracrRNA which is operably linked to the 3′ terminus of the direct repeat.
30. The method of embodiment 28 or 29, wherein the method further comprises introducing a polynucleotide encoding the type II Cas RNA dependent endonuclease (RDE) and optionally a polynucleotide comprising a DNA donor template into the cell.
31. A method of constructing a recombinant DNA molecule encoding a precursor RNA molecule comprising insertion of a first DNA molecule comprising SEQ ID NO: 1, 2, 3, or a conservatively substituted variant thereof which encodes a ribozyme with autocatalytic RNA cleavage activity at the 3′ end of a sense strand of a second DNA molecule encoding a product RNA molecule to obtain a recombinant DNA molecule encoding the precursor RNA molecule, wherein the product RNA molecule is heterologous to the ribozyme and is operably linked to the ribozyme in the precursor RNA molecule.
32. A method of producing an RNA with a defined 3′ terminus in a host cell comprising introducing into the host cell a recombinant DNA comprising a promoter which is operably linked to DNA encoding a precursor RNA molecule comprising from 5′ to 3′ a product RNA molecule which is operably linked at its 3′ end to a 3′-terminal ribozyme comprising an RNA encoded by SEQ ID NO: 1, 2, 3, or a conservatively substituted variant thereof having autocatalytic RNA cleavage activity, wherein the product RNA molecule is heterologous to the 3′-terminal ribozyme and wherein the product RNA is produced in the host cell.
33. The method of embodiment 31 or 32, wherein the recombinant DNA further comprises DNA encoding a hammerhead ribozyme or a hepatitis delta virus (HDV) ribozyme which is operably linked to the 5′ end of the DNA encoding the product RNA molecule.
34. The method of any one of embodiments 31, 32, or 33, wherein the product RNA comprises an siRNA, an miRNA, an aptamer, or a viral RNA replicon.
35. The method of any one of embodiments 31, 32, or 33, wherein the product RNA molecule comprises a guide RNA for a Cas RNA dependent endonuclease (RDE).
36. The method of any one of embodiments 31, 32, or 33, wherein the product RNA comprises a guide RNA for a type V Cas RNA dependent endonuclease (RDE), the guide RNA comprising from its 5′ to 3′ end a direct repeat (DR) element which can be bound by the type V Cas RDE and a spacer element which is operably linked to the DR.
37. The method of embodiment 36, wherein the precursor RNA molecule comprising the guide RNA and the 3′-terminal ribozyme further comprises at its 5′ end a hammerhead ribozyme.
38. The method of any one of embodiments 31, 32, or 33, wherein the product RNA comprises a guide RNA for a type II Cas RNA dependent endonuclease (RDE), the guide RNA comprising from its 5′ to 3′ end a spacer element and direct repeat (DR) element which can be bound by the type II Cas RDE and which is operably linked to the spacer element.
39. The method of embodiment 38, wherein the guide RNA is a single gRNA and wherein the product RNA further comprises a tracrRNA which is operably linked to the 3′ terminus of the direct repeat.
40. The method of embodiment 38 or 39, wherein the precursor RNA molecule comprising the guide RNA and the 3′-terminal ribozyme further comprises at its 5′ end a hammerhead ribozyme or a hepatitis delta virus (HDV) ribozyme.

### EXAMPLES

### Example 1. Description of Nucleic Acids and Expression Constructs Used

Nucleic acids used or described herein are set forth in Table 1.

TABLE 1
Nucleic Acids

| Description | SEQ ID NO |
|---|---|
| DNA encoding the Ht2 ribozyme | 1 |
| DNA encoding the Pmar-1 ribozyme | 2 |
| DNA encoding the Agam1-3 ribozyme | 3 |
| DNA encoding the CR1808 spacer of gRNA | 4 |
| DNA encoding the DR Element of gRNA | 5 |
| DNA encoding the DR-CR1808 spacer guide RNA | 6 |
| DNA encoding the 5′ HH ribozyme | 7 |
| DNA encoding the HDV ribozyme | 8 |

Expression vectors used in the experiments are described in Table 2. The following T-DNA plasmids were made with the respective expression cassettes within the left-border (LB) and right border (RB) region of the Agrobacterium-mediated plant transformation vectors. Expression constructs having the HH ribozyme at the 5′ terminus of the guide RNA also have a 6 Bp sequence aaatta which encodes a 6 base reverse complement (“6bpRC”) to the DR element of the guide RNA and which is operably linked to the 5′ end of the DNA encoding the HH ribozyme. The 6 base reverse complement (“6bpRC”) provides for proper folding and cleavage at the 5′ end of the DR element.

TABLE 2
Expression vectors

| | | Experimental |
|---|---|---|
| Plasmid | Description of expressed sequences | Description |
| pIN3608 | Cas-encoding -- AtU6-26p::CR938::polyT -- GFP- | AmpSeq (—) Control |
| | encoding | |
| pIN4549 | EPSPS-encoding -- Cas-encoding -- Pol IIp::DR- | DR 3′ guide processing |
| | CR1808-DR::Pol IIt -- mScarlet-encoding | |
| pIN4550 | EPSPS-encoding -- Cas-encoding -- Pol IIp::DR- | No 3′ guide processing |
| | CR1808::Pol IIt -- mScarlet-encoding | (experimental (—) control) |
| pIN4551 | EPSPS-encoding -- Cas-encoding -- Pol | 3′ HDV guide processing |
| | IIp::6bpRC HH-DR-CR1808-HDV::Pol IIt -- | (control) |
| | mScarlet-encoding | |
| pIN4552 | EPSPS-encoding -- Cas-encoding -- Pol | 3′ Ht2 (SEQ ID NO: 1) |
| | IIp::6bpRC HH-DR-CR1808-Ht2::Pol IIt -- | guide processing (Test 1) |
| | mScarlet-encoding | |
| pIN4553 | A EPSPS-encoding -- Cas-encoding -- Pol | 3′ Pmar-1 (SEQ ID NO: 2) |
| | IIp::6bpRC HH-DR-CR1808-Pmar-1::Pol IIt -- | guide processing (Test 2) |
| | mScarlet-encoding | |
| pIN4554 | EPSPS-encoding -- Cas-encoding -- Pol | 3′ Agam1_3 (SEQ ID NO: 3) |
| | IIp::6bpRC HH-DR-CR1808-Agam1_3::Pol IIt -- | guide processing (Test 3) |
| | mScarlet-encoding | |

### Example 2. Experimental results

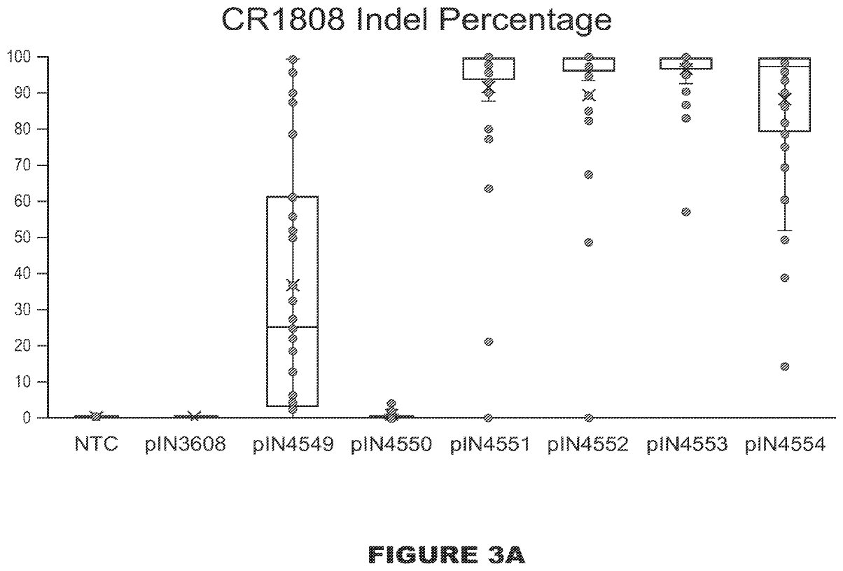

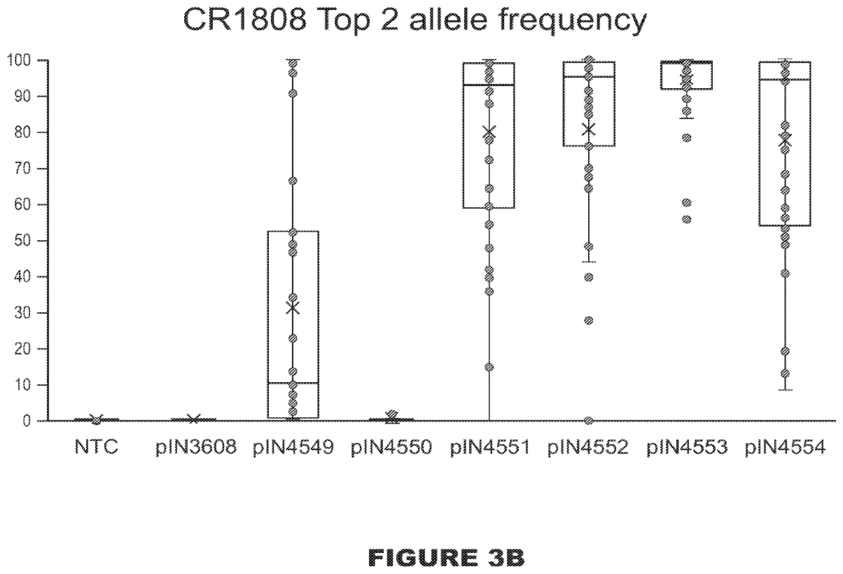

Plasmids set forth in Table 2 were electroporated into Rhizobium rhizogenes. Soybean hairy root transformation was performed with each construct essentially as described in Chen et al. (2017: doi.org/10.1016/j.cj.2017.08.006). Transformed samples were collected and genomic DNA extracted. The target locus from the transformed samples was sequenced to detect genomic edits induced by the DR-CR1808 spacer guide RNA. The graphs set forth in FIG. 3 summarize the edits detected.

Transformation and AmpSeq negative controls (NTC and pIN3608 which has a different crRNA) were at zero editing at the locus targeted by the gRNA, as expected. There are 2 different numbers derived from the AmpSeq results shown in FIG. 3. One is the overall insertion/deletion (indel) percentage. This is the frequency of indels at the locus targeted by the gRNA in the sequence pool and is shown in the left-most panel of FIG. 3. The frequency of the top 2 alleles (i.e., the two most frequent indels observed from sequencing) is shown in the right-most panel of FIG. 3. A high number in total indel percentage as shown in the left-most panel of FIG. 3 could come from continuous editing resulting in chimeric plants. A high number in the top 2 allele frequency shown in the right-most panel of FIG. 3 typically means the edit occurred early in development leading to the tissue being homogenous and the resultant plants are more likely to contain heritable edits. The increase in variability in some of the ribozyme architectures may suggest the edits are occurring a bit later, thus allowing for more alleles to occur in the tissue. Even so, there are still plenty of individuals with ~100% top 2 alleles, meaning there are plenty of individuals showing early editing. These numbers are often considered in the context of the likelihood of recovering individual plants with heritable edits.

The no guide RNA processing control vector pIN4550 showed no editing. This confirms the need for the crRNA to be properly processed for editing to occur.

The pIN4549 vector which relies on Cas-mediated DR processing of the precursor RNA behind a Pol II promoter had variable levels of editing. Total indel frequency shows an average of ~35% editing. Half of the edits fall within the range of 5-60% editing. With the results of the other treatments, this variability and low editing suggests improper or incomplete processing of the guide RNA.

The pIN4551 (HDV 3′-terminal ribozyme), pIN4552 (SEQ ID NO: 1 as 3′-terminal ribozyme), pIN4553 (SEQ ID NO: 2 as 3′-terminal ribozyme), and pIN4554 (SEQ ID NO: 3 as 3′-terminal ribozyme) vectors were all the ribozyme containing constructs which performed well for total indel frequency. pIN4551, pIN4552, and pIN4553 had almost 100% editing in all of the samples. When looking at just the top 2 allele frequencies, pIN4551 and pIN4552 had a decrease in editing and an increase in variability. pIN4553 maintained a high level of editing with barely any increase in variability. From these results, constructs containing the Ht2 3′-terminal ribozyme (SEQ ID NO: 1) and Pmar-1 3′-terminal ribozyme seem to have the highest editing efficiency.

What is claimed is:

1. A DNA molecule comprising from 5′ to 3′ of its sense strand a promoter which is operably linked to a DNA molecule encoding a precursor RNA molecule comprising from its 5′ to 3′ end a product RNA molecule which is operably linked at its 3′ end to a 3′-terminal ribozyme comprising an RNA encoded by SEQ ID NO: 1, 2, 3, or a conservatively substituted variant thereof having autocatalytic RNA cleavage activity, wherein the product RNA molecule is heterologous to the 3′-terminal ribozyme.

2. The DNA molecule of claim 1, wherein the product RNA molecule comprises a guide RNA for a Cas RNA dependent endonuclease (RDE).

3. The DNA molecule of claim 1, wherein the product RNA molecule comprises a guide RNA for a type V Cas RNA dependent endonuclease (RDE), the guide RNA comprising from its 5′ to 3′ end a direct repeat (DR) element which can be bound by the type V Cas RDE and a spacer element which is operably linked to the DR.

4. The DNA molecule of claim 3, wherein the precursor RNA molecule comprising the guide RNA and the 3′-terminal ribozyme further comprises at its 5′ end a hammerhead ribozyme which is operably linked to the guide RNA.

5. The DNA molecule of claim 1, wherein the precursor RNA molecule comprises from its 5′ to 3′ end a hammerhead ribozyme, the guide RNA comprising a direct repeat (DR) element which can be bound by a type V Cas RDE a spacer element, and the 3′-terminal ribozyme, wherein the hammerhead ribozyme and the 3′-terminal ribozyme are operably linked to the guide RNA.

6. The DNA molecule of claim 1, wherein the product RNA molecule comprises a guide RNA for a type II Cas RNA dependent endonuclease (RDE), the guide RNA comprising from its 5′ to 3′ end a spacer element and direct repeat (DR) element which can be bound by the type II Cas RDE and which is operably linked to the spacer element.

7. The DNA molecule of claim 6, wherein the precursor RNA molecule comprising the guide RNA and the 3′-terminal ribozyme further comprises at its 5′ end a hammerhead ribozyme or a hepatitis delta virus (HDV) ribozyme which is operably linked to the guide RNA.

8. The DNA molecule of claim 6, wherein the guide RNA is a single gRNA wherein the product RNA further comprises a tracrRNA which is operably linked to the 3′ terminus of the direct repeat.

9. The DNA molecule of claim 1, wherein the product RNA molecule comprises an siRNA, an miRNA, an aptamer, or a viral RNA replicon.

10. The DNA molecule of claim 9, wherein the precursor RNA molecule further comprises at its 5′ end hammerhead ribozyme or a hepatitis delta virus (HDV) ribozyme which is operably linked to the product RNA molecule.

11. The DNA molecule of claim 1, wherein the DNA molecule further comprises at the 3′end of the sense strand a terminator element which is operably linked to the DNA encoding the precursor RNA molecule.

12. The DNA molecule of claim 1, wherein the promoter is recognized by a eukaryotic RNA Polymerase II, optionally wherein the eukaryotic RNA Polymerase II is a monocot or dicot plant RNA Polymerase II or optionally wherein the eukaryotic RNA Polymerase II is a mammalian RNA Polymerase II.

13. The DNA molecule of claim 1, wherein the promoter is recognized by a eukaryotic RNA Polymerase III, optionally wherein the eukaryotic RNA Polymerase III is a monocot or dicot plant RNA Polymerase III or optionally wherein the eukaryotic RNA Polymerase III is a mammalian RNA Polymerase III.

14. A cell comprising the DNA molecule of any one of claims 1 to 13.

15. The cell of claim 14, wherein the cell is a bacterial cell, a plant cell, or a mammalian cell.

16. The cell of claim 15, wherein the bacterial cell is an Agrobacterium, Rhizobium sp., Sinorhizobium sp., Mesorhizobium sp., Bradyrhizobium sp., Azobacter sp., or Phyllobacterium sp. and optionally wherein the DNA molecule further comprises the promoter and the terminator recognized by the eukaryotic RNA Polymerase II or RNA Polymerase III and optionally wherein the eukaryotic RNA Polymerase II or RNA Polymerase III is the monocot or dicot plant RNA Polymerase II or RNA Polymerase III.

17. The cell of claim 15, wherein the cell is a monocot or a dicot plant cell, optionally wherein the DNA molecule further comprises the promoter and the terminator recognized by a eukaryotic RNA Polymerase II or RNA Polymerase III and optionally wherein: (i) the eukaryotic RNA Polymerase II or RNA Polymerase III is a monocot plant RNA Polymerase II or RNA Polymerase III and the cell is a monocot plant cell; or (ii) optionally wherein the eukaryotic RNA Polymerase II or RNA Polymerase III is a dicot plant RNA Polymerase II molecule or RNA Polymerase II and the cell is a dicot plant cell.

18. The cell of claim 15, wherein the cell is a mammalian cell, optionally wherein the DNA molecule further comprises the promoter and the terminator recognized by a mammalian RNA Polymerase II or RNA Polymerase III and optionally wherein the mammalian cell is not directly obtained from a human embryo.

19. A method of delivering a product RNA to a cell comprising the step of introducing the DNA molecule of any one of claims 1 to 13 into the cell.

20. The method of claim 19, wherein the cell is a monocot or a dicot plant cell, optionally wherein the DNA molecule further comprises the promoter and the terminator recognized by a eukaryotic RNA Polymerase II or RNA Polymerase III and optionally wherein: (i) the eukaryotic RNA Polymerase II or RNA Polymerase III is a monocot plant RNA Polymerase II or RNA Polymerase III and the cell is a monocot plant cell; or (ii) optionally wherein the eukaryotic RNA Polymerase II or RNA Polymerase III is a dicot plant RNA Polymerase II or RNA Polymerase III and the cell is a dicot plant cell.

21. The method of claim 19, wherein the cell is a mammalian cell, optionally wherein the DNA molecule further comprises the promoter and the terminator recognized by the mammalian RNA Polymerase II or RNA Polymerase III and optionally wherein the mammalian cell is not directly obtained from a human embryo.

22. The method of claim 19, wherein the product RNA comprises an siRNA, an miRNA, an aptamer, or a viral RNA replicon.

23. The method of claim 19, wherein the product RNA molecule comprises a guide RNA for a Cas RNA dependent endonuclease (RDE).

24. The method of claim 19, wherein the product RNA comprises a guide RNA for a type V Cas RNA dependent endonuclease (RDE), the guide RNA comprising from its 5′ to 3′ end a direct repeat (DR) element which can be bound by the type V Cas RDE and a spacer element which is operably linked to the DR, optionally wherein the precursor RNA molecule comprising the guide RNA and the 3′-terminal ribozyme further comprises at its 5′ end a hammerhead ribozyme.

25. The method of claim 24, wherein the method further comprises introducing a polynucleotide encoding the type V Cas RNA dependent endonuclease (RDE) into the cell.

26. The method of claim 24, wherein the method further comprises introducing a polynucleotide encoding the type V Cas RNA dependent endonuclease (RDE) and a polynucleotide comprising a DNA donor template into the cell.

27. The method of claim 24, where the precursor RNA comprises the 5′ hammerhead ribozyme and wherein the frequency of gene editing events is increased in comparison to a control comprising a DNA molecule comprising the promoter, the 5′ hammerhead ribozyme, the guide RNA, and a hepatitis delta virus (HDV) ribozyme which is operably linked to the 3′ terminus of the guide DNA in the control RNA molecule.

28. The method of claim 19, wherein the product RNA comprises a guide RNA for a type II Cas RNA dependent endonuclease (RDE), the guide RNA comprising from its 5′ to 3′ end a spacer element and direct repeat (DR) element which can be bound by the type II Cas RDE and which is operably linked to the spacer element, optionally wherein the precursor RNA molecule comprising the guide RNA and the 3′-terminal ribozyme further comprises at its 5′ end a hammerhead ribozyme or a hepatitis delta virus (HDV) ribozyme.

29. The method of claim 28, wherein the guide RNA is a single gRNA and wherein the product RNA further comprises a tracrRNA which is operably linked to the 3′ terminus of the direct repeat.

30. The method of claim 28, wherein the method further comprises introducing a polynucleotide encoding the type II Cas RNA dependent endonuclease (RDE) and optionally a polynucleotide comprising a DNA donor template into the cell.

31. A method of constructing a recombinant DNA molecule encoding a precursor RNA molecule comprising insertion of a first DNA molecule comprising SEQ ID NO: 1, 2, 3, or a conservatively substituted variant thereof which encodes a ribozyme with autocatalytic RNA cleavage activity at the 3′ end of a sense strand of a second DNA molecule encoding a product RNA molecule to obtain a recombinant DNA molecule encoding the precursor RNA molecule, wherein the product RNA molecule is heterologous to the ribozyme and is operably linked to the ribozyme in the precursor RNA molecule.

32. A method of producing an RNA with a defined 3′ terminus in a host cell comprising introducing into the host cell a recombinant DNA comprising a promoter which is operably linked to DNA encoding a precursor RNA molecule comprising from 5′ to 3′ a product RNA molecule which is operably linked at its 3′ end to a 3′-terminal ribozyme comprising an RNA encoded by SEQ ID NO: 1, 2, 3, or a conservatively substituted variant thereof having autocatalytic RNA cleavage activity, wherein the product RNA molecule is heterologous to the 3′-terminal ribozyme and wherein the product RNA is produced in the host cell.

33. The method of claim 31 or 32, wherein the recombinant DNA further comprises DNA encoding a hammerhead ribozyme or a hepatitis delta virus (HDV) ribozyme which is operably linked to the 5′ end of the DNA encoding the product RNA molecule.

34. The method of claim 31 or 32, wherein the product RNA comprises an siRNA, an miRNA, an aptamer, or a viral RNA replicon.

35. The method of claim 31 or 32, wherein the product RNA molecule comprises a guide RNA for a Cas RNA dependent endonuclease (RDE).

36. The method of claim 31 or 32, wherein the product RNA comprises a guide RNA for a type V Cas RNA dependent endonuclease (RDE), the guide RNA comprising from its 5′ to 3′ end a direct repeat (DR) element which can be bound by the type V Cas RDE and a spacer element which is operably linked to the DR.

37. The method of claim 31 or 32, wherein the precursor RNA molecule comprising the guide RNA and the 3′-terminal ribozyme further comprises at its 5′ end a hammerhead ribozyme.

38. The method of claim 31 or 32, wherein the product RNA comprises a guide RNA for a type II Cas RNA dependent endonuclease (RDE), the guide RNA comprising from its 5′ to 3′ end a spacer element and direct repeat (DR) element which can be bound by the type II Cas RDE and which is operably linked to the spacer element.

39. The method of claim 38, wherein the guide RNA is a single gRNA and wherein the product RNA further comprises a tracrRNA which is operably linked to the 3′ terminus of the direct repeat.

40. The method of claim 38, wherein the precursor RNA molecule comprising the guide RNA and the 3′-terminal ribozyme further comprises at its 5′ end a hammerhead ribozyme or a hepatitis delta virus (HDV) ribozyme.
